(19) United States
(12) Patent Application Publication
(10) Pub. No.: US 2011/0178466 A1
(43) Pub. Date: Jul. 21, 2011

(54) MEDICAL ATTACHMENT DEVICE

(75) Inventors: Mihai Vioreanu, Dublin (IE); John O'Byrne, Dublin (IE)

(73) Assignee: INGENIUM MEDICAL SOLUTIONS LIMITED, Dublin (IE)

(21) Appl. No.: 12/524,287

(22) PCT Filed: Jan. 28, 2008
(86) PCT No.: PCT/EP2008/050964

§ 371 Date: Mar. 10, 2011

(30) Foreign Application Priority Data
Jan. 26, 2007 (IE) S2007/0045

(51) Int. Cl. A61M 25/02; A61M 1/00; A61B 17/068

(52) U.S. Cl. 604/180; 604/174; 604/540; 227/175.1

(57) ABSTRACT

The present invention provides a medical attachment device for securing a catheter or drain to a patient comprising at least one medical staple capable of being anchored to the patient's skin tissue; and the medical staple is adapted to be coupled to a connector, wherein the connector attaches to the catheter or drain to secure the catheter or drain in a fixed position. In another embodiment the invention provides a medical attachment device for securing a catheter or drain to a patient comprising a base comprising a layer of adhesive, suitably made of hypoallergenic material, capable of being anchored to the patient's skin tissue through an adhesive mechanism and/or at least one medical staple through a plurality of openings provided in said base; and said base comprises an embedded connector for attaching to the catheter or drain and securing the catheter or drain in a fixed position. The invention provides provide a very effective safe and simple catheter securement device for attaching to patients.

### FIELD OF THE INVENTION

The invention relates to a medical attachment device and in particular to a medical drain/plastic tube (for example a central line or feeding tube) or catheter attachment device used in the medical or surgical field.

### BACKGROUND TO THE INVENTION

Surgical drains or catheters are commonly used medical devices used by healthcare workers (doctors, nurses etc) in most areas of hospital medicine (surgery, emergency medicine, radiology, oncology etc). Catheter placement is the most common invasive medical procedure. For example in the US alone, more then 300 million peripheral intravenous catheters are sold each year, according to industry analysts, plus millions more central venous, arterial, urinary and other types of surgical catheters.

Currently the surgical catheter is secured by stitching it to the patient's skin, using a sharp needle and tying the suture around the catheter a few times and finally tying a knot around the catheter without obstructing the drain. A problem with this approach is that it is a time consuming procedure requiring a specific skill (stitching, knotting technique) with a recognised infection or inoculation risk for the surgeon/anaesthetist. Suture needles are the main source of needle stick injuries for healthcare workers, causing 51 percent of all sharp injuries in surgical settings. Research published in US indicates that the risk of accidental needle stick from suture needles during the catheter securement process is consistently more than 1 percent (that is, one potential exposure to bloodborne pathogens per one hundred catheters secured), and as high as 2 percent (Journal of Vascular and Interventional Radiology, January 2002). Indeed according to some anecdotal hospital reports the risk can be in excess of 5 percent.

It is estimated that Bloodstream Infections (BSI) associated with catheters kill as many as 20,000 American citizens a year, and cost up to US$2.3 billion annually to treat. The Centre for Disease Control and Prevention (CDC) suggests that suture securement of catheters is a significant cause for these infections. According to the latest CDC Guidelines “Sutureless securement devices can be advantageous over suture in preventing catheter related BSIs” [SeeGuidelines for preventing intra-vascular related infections' http://www.cdc.gov/mmwr/preview/mmwrhtml/rr5110a1.htm].

According to the American Hospital Association, one case of serious infection by bloodborne pathogens can add up to $1 million or more in expenditures for testing follow-up, lost time and disability payments. The cost of follow-up of a percutaneous injury is between $500 and 2500 (if no infection is contacted). For the estimated of 54,000 injuries caused by suture needles each year, that translates to a potential cost to to US hospitals of $36.3 million per year.

One type of device to overcome the above mentioned problems is disclosed in U.S. Pat. No. 5,730,758 in the name of Allgeyer and discloses a staple and staple applicator for use in skin fixation of catheters. However a problem with this device is that it is device specific and requires a built in hub. The device relates to securing of vascular drains only and does not allow flexibility in the length attachment of the inserted drain and is difficult to use.

An alternative to suture securement is applying adhesive or adhesive tape to hold the catheter in place. However, both published and unpublished data suggest a failure rate of 65 percent and a contamination of 74 percent when tape is used to secure surgical catheters. There are some adhesive attaching devices but they are not so popular. Adhesive material can be an allergen/irritant substance for the patient's skin, and the adhesion is not secure after 24 hours. In addition adhesive materials require daily check and could be uncomfortable to remove. Moreover the adhesive may be too strong and can be difficult to remove and be very sore for the patient when removing the adhesive. Overall the use of adhesive tape has been found not to provide a satisfactory solution.

It is an object of the invention to provide a device for securing a catheter or drain to a patients skin to overcome the above mentioned problems.

### SUMMARY OF THE INVENTION

According to the invention there is provided, as set out in the appended claims, a medical attachment device for securing a catheter or drain to a patient comprising: a medical staple capable of being anchored to the patients skin tissue; and the medical staple is adapted to be coupled to a connector for attaching to the catheter or drain and securing the catheter or drain in a fixed position.

The inventive sutureless catheter securement device provides a simple, fast way of attaching a medical catheter to the patient's skin. The drain attachment is sterile, of single use, does not require a specific skill to be applied. The drain attachment is more comfortable for the patient and virtually eliminates infection or inoculation risk to healthcare workers. The drain attachment can be removed very easily. The inventive attachment eliminates the risk of irritability or allergies and eliminates the risk of needle stick injuries and the associated costs.

Suitably, the connector comprises a tube attachment for receiving the catheter or drain to secure the catheter or drain in a fixed position. The tube attachment may comprise an adjustable opening for receiving the catheter or drain.

Suitably, the opening comprises an adjustment mechanism, such as a ratchet mechanism for adjusting the opening to securely fasten the tube attachment around the catheter or drain. Preferably the ratchet mechanism comprises a plurality of holes and buttons, wherein the buttons can snap through the holes to secure the catheter or drain in the fixed position.

Suitably, the inner surface of the opening comprises teeth for gripping the catheter or drain in a fixed position.

Suitably, the adjustable opening comprises a substantially circular cross sectional area. The connector can be made of a plastic type or silicon material.

Suitably, the staple is a skin clip, optionally of the metal type and comprises means for penetrating skin tissue. The staple can be made from a biocompatible and/or hypoallergenic material. The connector can be made from a biocompatible and/or hypoallergenic material.

Suitably part of the connector comprises at least one slot for receiving one leg of said staple to anchor the staple to the patients skin tissue and securing the connector in a fixed position. The connector may be provided with two slots, a first slot for receiving one leg of said staple and a second slot for receiving a second leg of said staple to anchor the staple to the patients skin tissue.

Alternatively the connector may be provided with two slots, a first slot for receiving a leg of a first staple and a second slot for receiving a leg of a second staple such that said first and second staples anchor the connector to the patients skin tissue.

Suitably, the surgical staple and the connecter are integrally formed.

In another embodiment of the invention there is provided a method for securing a catheter or drain to a patient comprising the steps of:
stapling a staple to a patients skin tissue to anchor the staple; coupling the staple to a connector; and
attaching the catheter or drain to the connector to secure the catheter or drain in a fixed position.

In a further embodiment of the invention there is provided a kit comprising a medical stapler and a medical attachment device for securing a catheter or drain to a patient comprising: a medical staple capable of being anchored to the patients skin tissue; and the medical staple is adapted to be coupled to a connector for attaching to the catheter or drain and securing the catheter or drain in a fixed position.

In another preferred embodiment of the present invention there is provided medical attachment device for securing a catheter or drain to a patient comprising a base comprising a layer of adhesive, suitably made of hypoallergenic material, capable of being anchored to the patients skin tissue through an adhesive mechanism and/or at lease one medical staple through a plurality of openings provided in said base; and said base comprises an embedded connector for attaching to the catheter or drain and securing the catheter or drain in a fixed position.

The provision of both adhesive and the staple/skin clip attachment for securing the device is very advantageous over existing catheter securing devices. Suitably, the adhesive base of the attachment device that will be applied onto the patient skin has an adhesive surface to stick to the patient skin. The adhesive base can be oval shaped and effectively provides an adhesive mechanism for anchoring the base to a patients skin tissue. The adhesive surface is covered by a removable layer and can be made of soft, hypoallergenic material. This layer is removed by the applicator (for example a healthcare provider) prior to sticking the device to the patient skin. If the applicator's intention is not to use the adhesive method for securement, the soft layer can be left unpeeled and the device attached to the patient skin with staples or skin clips through the openings provided.

Suitably, the embedded connector comprises a channel for receiving the catheter or drain to secure the catheter or drain in a fixed position through its adhesive mechanism. The channel may comprise an adjustable opening for receiving the catheter or drain that also permits closing of the channel to secure the drain to the base. It will be appreciated that the channel may comprise a snap fit connection for securing the drain to the base.

Suitably, the base comprises an adjustment mechanism, such as two adhesive flaps that stick to the drain, being stretched in opposite directions and over the channel, and finally secured to the base to securely fasten the drain to the base.

Suitably, the inner surface of the channel comprises a coated adhesive material for holding the catheter or drain in a fixed position. The adhesive material is uncovered, before securing the tube by peeling off a transparent, detachable thin plastic layer. This is particularly advantageous for securing the catheter or drain.

Suitably, the flaps are covered on their inner surface with adhesive material for sticking onto the drain (which is already slotted into the adhesive opening/imbedded connector) and then sticking onto the base. The orientation of the two flaps can be 180 degree opposite to each other, and their elastic properties and the adhesive mechanism put in place secures the drain/catheter in the desired position, preventing it to slide in and out of the body. The flaps can be made of an elastic type material so that the elastic properties can be placed at any angle to each other to secure the drain to the base.

Suitably, the adjustable opening or channel comprises a substantially circular cross sectional area. The base and imbedded connector and flaps can be made of a plastic type or silicon material.

In one embodiment the oval base of the attachment device comprises eight openings evenly distributed around its periphery for securement to the skin using an existent skin clip that penetrates the skin tissue with one arm piercing through the opening to further secure the attachment device to the skin. Using the skin clip is optional to the adhesive mechanism provided onto the whole surface of the oval base that comes applied directly onto the skin. It will be appreciated that a user is expected to use as many skin clips through the openings provided as he/she feels is necessary to sufficiently secure the attachment device to the skin. Typically using two skin clips through the openings directly opposite to each other on the long axis of the oval base provides for secure attachment of the device to the patients skin.

### BRIEF DESCRIPTION OF THE DRAWINGS

The invention will be more clearly understood from the following description of an embodiment thereof, given by way of example only, with reference to the accompanying drawings, in which:—

FIG. 1a & 1b illustrates a stapler incorporating a surgical attachment device according to one aspect of the invention.

FIG. 2 shows the surgical attachment device according to the present invention.

FIG. 3 shows the surgical attachment device according to another embodiment of the present invention.

FIG. 4 shows the surgical attachment device according to a further embodiment of the present invention.

FIGS. 5 and 5a shows a connector sleeve of the surgical attachment device.

FIG. 6 illustrates an example of the inner surface of the connector sleeve of FIG. 5.

FIGS. 7a-7e illustrate various examples of a connection between the connector sleeve and a surgical staple.

FIG. 8 illustrates an embodiment of the invention in use.

FIGS. 9 to 13 illustrate side, plan, rear and perspective views of the surgical attachment device according to another embodiment of the present invention

### DETAILED DESCRIPTION OF THE DRAWINGS

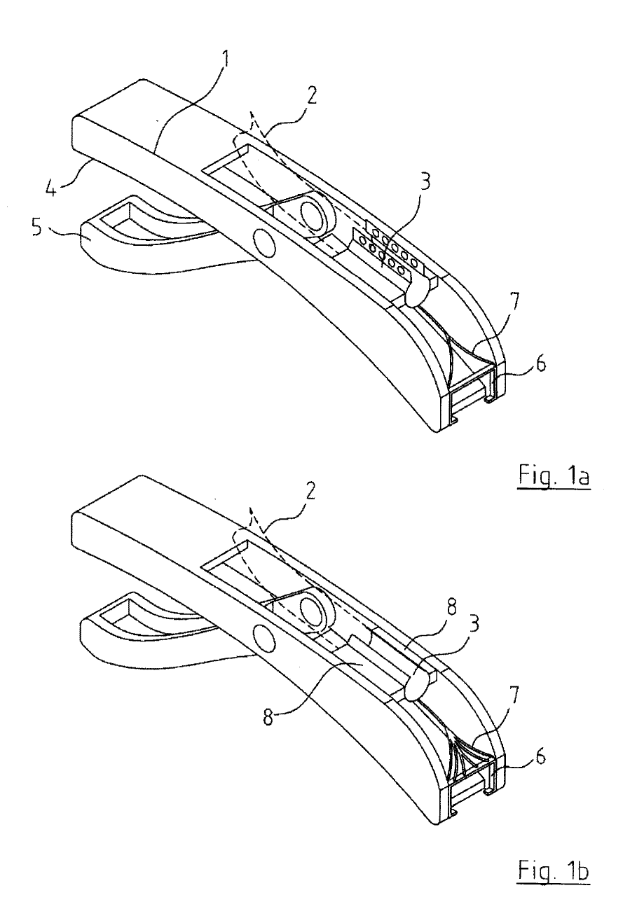

Referring now to FIG. 1a & 1b there is shown a staple applicator 1, for example a medical or surgical stapler. The staple applicator 1 is adapted to receive a medical attachment device 3, 6, 7, according to the invention. The staple applicator comprises a pair of handles 4 and 5 to be held between the thumb and index fingers of a user to allow for easy application of the surgical attachment device 3, 6, 7 to be anchored to a patients skin tissue (not shown).

The medical attachment device comprises a surgical staple 6 and is adapted to be coupled to a connecter portion 3, 7. The connecter portion 3, 7 can be in the form of a sleeve 3 and may be attached to the surgical staple 6 by a flexible material type portion 7.

The staple applicator 1 can be provided with a groove or channel 2. The sleeve 3 can sit in the groove or a channel 2 before use. The staple applicator 1 has a side-to-side compression device 8 to close around the sleeve 3 securely before use, for example a spring loaded compression mechanism. The surgical attachment device 3, 6, 7 is ready for securing a catheter or drain to a patient.

In operation a user can use the staple applicator 1 by stapling the surgical attachment device to the patients skin at a desired location. This can be made possible by pinching the patient's skin to provide a suitable target area for applying the staple. Once the staple 6 has been applied and penetrates the patients skin tissue to anchor to the patients skin tissue, the sleeve 3 can be peeled away from the staple applicator 1. It will be appreciated that the surgical staple 6 can be a metal type skin clip comprising means for penetrating skin tissue, which may take the form of one or more legs substantially pointed to penetrate the skin tissue. The catheter or drain (not shown) can be inserted in the sleeve 3 and secured so that the catheter or drain is secured in a fixed position to the patients skin. The invention provides a simple method for securing a catheter or drain to a patient by performing the steps of: stapling a staple to a patients skin tissue to anchor the staple; coupling the staple to a connector; and attaching the catheter or drain to the connector to secure the catheter or drain in a fixed position.

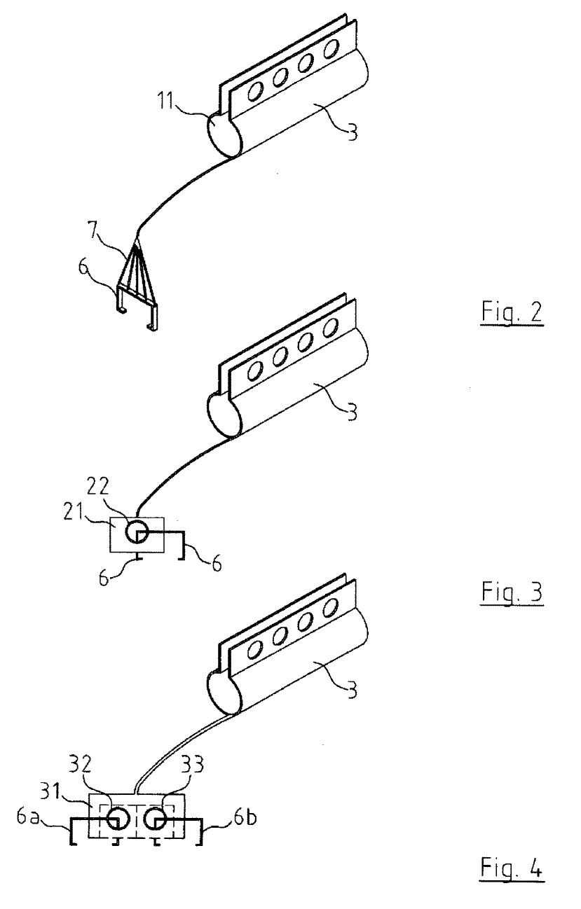

Referring now to FIG. 2 illustrates the medical attachment device according to one embodiment of the present invention wherein the sleeve 3 comprises a substantially hollow tubular attachment for receiving the catheter or drain to secure the catheter or drain in a fixed position. The sleeve 3 defines an adjustable opening 11 which has a substantially circular cross section area, but not limited to such, for receiving the catheter or drain. The sleeve 3 can be connected to the staple 6 for example using a plastic strap connection 7. The plastic strap connection 7 can be integrally formed with the medical staple 6. In another embodiment the sleeve 3, the plastic strap connection and the medical staple 6 can be integrally formed with each other.

Referring to FIG. 3 there is illustrated another embodiment of the surgical attachment device. At one end of the plastic strap connection 7 there is provided an area 21 having at least one slot 22. In operation the surgical staple 6 can be stapled to the patients skin, as described above, wherein the slot 22 receives one leg 6 of the staple 6 to anchor the staple 6 to the patients skin tissue to secure the sleeve 3 in a fixed position, and thereby securing the catheter or drain in a fixed position. An alternative embodiment is shown in FIG. 4 wherein one end of the plastic strap connection 7 is provided an area 31 having two (or more) slots 32 and 33 each for receiving a leg of two different surgical staples 6. It will be appreciated that a single staple can be used through the two slots or alternatively to increase the strength of the anchor two staples are used 6a and 6b wherein one leg of each staple anchors the connector 31 to the patients skins tissue for added strength. It will be further appreciated that more that two slots can be used, for example four slots could be used for a chest drain.

It will be appreciated that the connecter can be made from a biocompatible or hypoallergenic material. Ideally the medical staple 6 and the connecter 3 are integrally formed with each other.

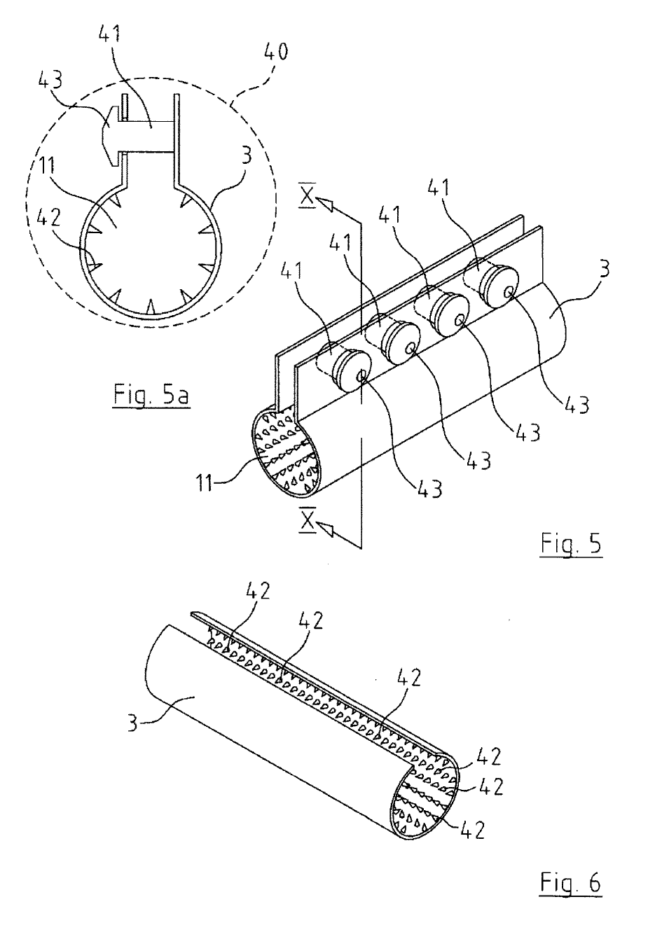

Referring to FIGS. 5 and 6 the connecter sleeve 3 is shown in more detail. The sleeve 3 may comprise a substantially tubular shape for receiving a catheter or drain (not shown) to secure the catheter or drain in a fixed position. This is achieved by providing an adjustable opening 11 for receiving the catheter or drain tube. In addition FIG. 5a shows a cut through section view XX of the substantially tubular member 3, indicated by the reference numeral 40. The size of the substantially circular opening 11 can be variable in size depending on the drain or catheter diameter. The inner surface of the sleeve 3 is provided with a number of teeth or ridges 42 to provide a gripping or frictional surface to secure the catheter or drain tube in a fixed position. The adjustable opening 11 is provided with a means for adjusting the size of the opening to securely fasten the tubular attachment around the catheter or drain. This can be achieved for example using a number of holes and buttons wherein the buttons 43 snap through the holes 41 to securely fasten the sleeve 3 around the catheter or tube. Alternatively a ratchet mechanism for adjusting the opening to securely fasten the tubular attachment around the catheter or drain can be employed.

It will appreciated that the diameter of the sleeve when in a closed position is marginally less than the outer diameter of catheter to be secured to ensure a tight grip of the tube without damaging the catheter or drain. Referring to FIG. 6 illustrates the internal surface of the sleeve connecter portion 3 in more detail. The internal surface comprises a number of teeth 42 to increase the frictional grip when securing the catheter or drain in a fixed position.

It is a envisaged that the sleeve 3 will be in region of 1.5 to 2.5 cm long bearing in size depending on the size of the catheter to be fixed to the patient's tissue. It will be appreciated the sleeve is made from a rigid type material to prevent the catheter to slide in and out from the body.

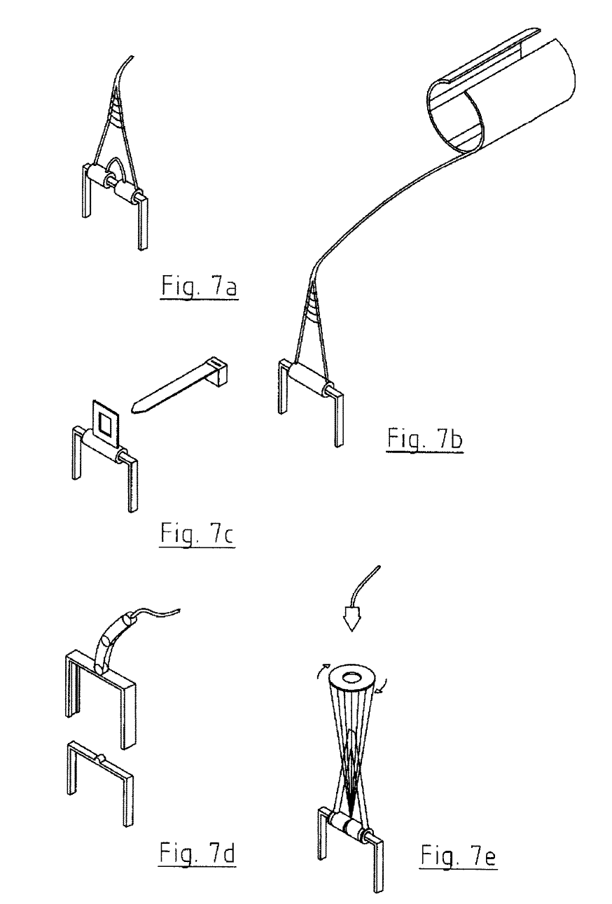

Referring to FIG. 7 illustrates a number of different types of attachments for securing the staple to the sleeve portion 3. FIGS. 7a and 7b illustrate how a plastic, or other type of material, moulding is designed to attach securely to the staple. FIG. 7c illustrates a strap arrangement wherein the staple is adapted to receive a strap to secure to the sleeve portion 3. FIGS. 7d and 7e show alternative arrangements of securing the sleeve to the staple. It will be further appreciated that the connecter sleeve is made of a plastic type or silicon type material.

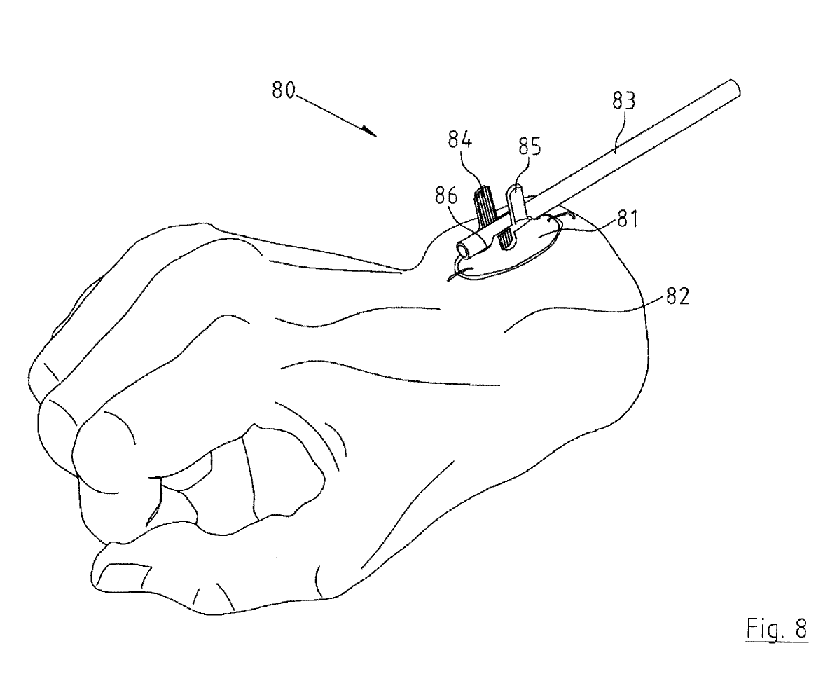

Referring now to FIGS. 8 to 13 inclusive, and initially FIG. 8 shows a device according to a further embodiment of the present invention, indicated generally by the reference numeral 80. The device 81 can be attached to a patients skin 82 and comprises a pair of flaps 84 and 85 for securing a catheter tube or drain 83 in a channel 86 of the device 81.

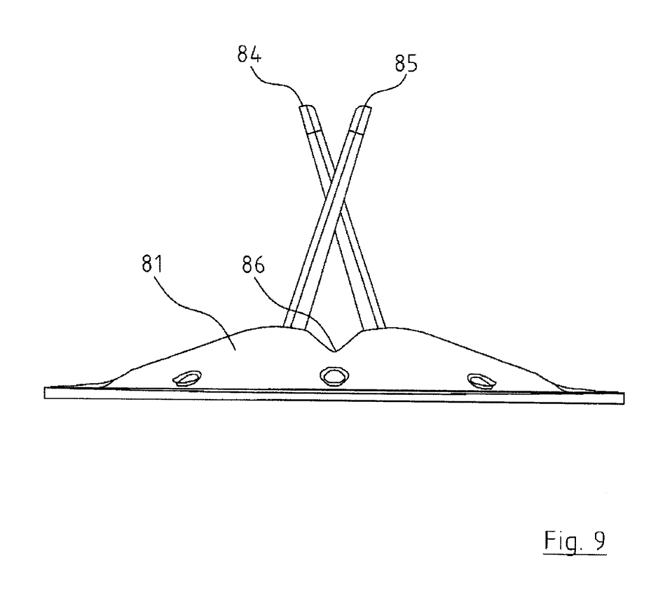

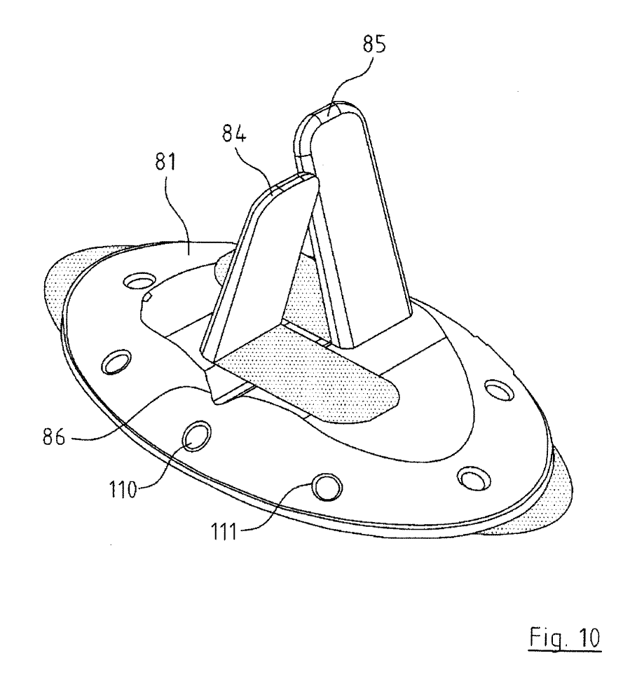

Referring to FIGS. 9 and 10, there is shown the device shown of FIG. 8 in a side and perspective views. The device 81 has a substantially oval shaped base. It will be appreciated that the device can be of any suitable shape. The periphery of the oval shaped base is provided with a number of slots or openings 110 and 111. The underside of the base is provided with a layer of hypoallergenic adhesive material. The layer of adhesive can be protected by a thin layer of protective material, for example paper, which may be peeled off before use. The pair of flaps 84 and 85 can also be provided with a layer of adhesive that can be moved into a locking position to secure the catheter tube 83 in use. The base 81 anchors or sticks to the patients skin in use. The base 81 is elevated and tapered out towards its periphery such that an opening is embedded in the base 81 to provide the channel 86 for receiving the catheter drain. The channel 86 is provided with a coating or layer of adhesive to grip or anchor the catheter in use. This feature is very advantageous to anchor the catheter tube or drain securely.

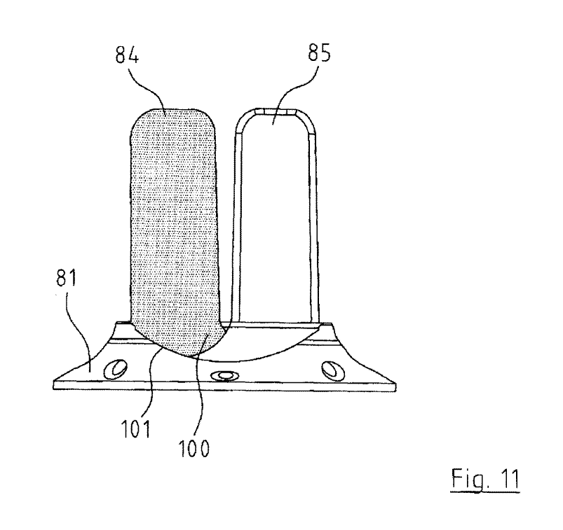

Referring to FIG. 11, the tapered portion of the oval base is partly provided with an adhesive 100 and 101 and covered by a thin transparent plastic layer. This layer can be easily peeled off before use. The inner part of the flaps 84 and 85 is also provided with a layer of adhesive such that the flaps engage the adhesive on the base 81 to further secure the catheter tube or drain 83.

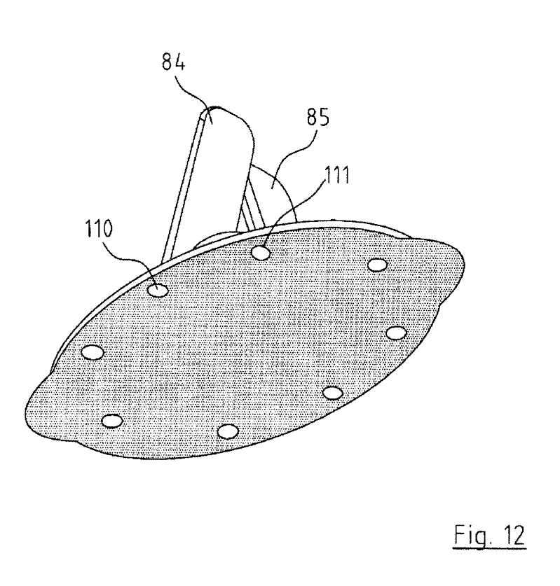

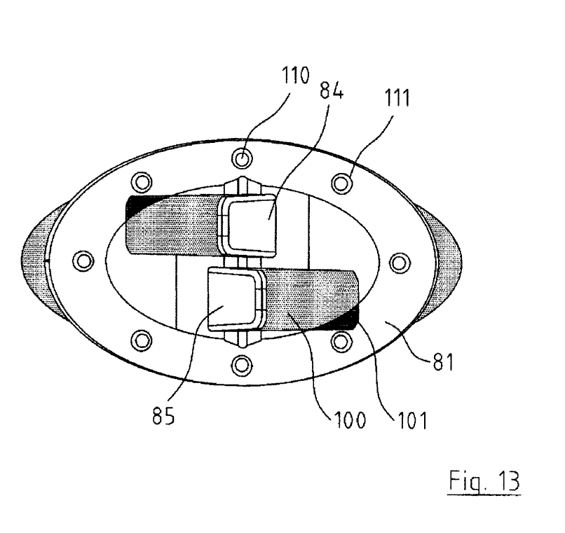

FIGS. 12 and 13 shows a perspective view and plan of the underside of the oval base 81. The outer periphery of the base is provided with a number of slots openings 110 and 111. As can be seen in FIG. 11, the underside is provided with a layer of adhesive that is protected by a layer of protective material, that can easily be peeled off before use. In addition there is provided a gripping portion or peeling tab at both ends of the long axis of the oval base 81 to allow for easily peeling off of the protective layer of hypoallergenic material.

The tapered portion of the base is laid with an adhesive and covered by a thin transparent plastic layer as shown by reference numeral 100. At the end of these portions, it is provided with a single tab (or two tabs, one positioned at each end) to provide a gripping part of the detachable layer 101 for peeling off the layer of protective material before use.

It will be appreciated that a combination of the adhesive for the base to attach to the patients skin along with the flaps provides a simple attachment mechanism. In addition, the attachment procedure is further complimented by the openings (holes) that provide for the staples or skin clips. It will be further appreciated that the layer of adhesive provided in the channel 86 provides a simple way of securing the catheter tube to the device. It will be appreciated that the flaps 84 and 85 are made from a resilient type material to securely hold the tube in place. It will be appreciated that this embodiment can be used in conjunction with the embodiment of FIGS. 1-7 to provide a very effective safe and simple catheter securement device.

It will be appreciated that the device of the present invention can be provide in any shape or size and adapted to accommodate different sized drains, for example central lines, chest drains, etc that are used tin the medical field.

It will be further appreciated that the present description describes the use of catheters or drains generally and encompasses any type of medical tubing inserted into a body, for example cannulae or medical tubing used in conjunction with a trocar which are well known in the medical field. It will also be appreciated that the present invention can be easily employed in the field of veterinary and used for securing a drain to animals.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The invention is not limited to the embodiments hereinbefore described but may be varied in both construction and detail.

1. A medical attachment device for securing a catheter or drain to a patient comprising:
at least one medical staple capable of being anchored to the patient's skin tissue; and
the medical staple is adapted to be coupled to a connector, wherein the connector attaches to the catheter or drain to secure the catheter or drain in a fixed position.

2. The device of claim 1 wherein the connector comprises a substantially tubular attachment for receiving the catheter or drain to secure the catheter or drain in a fixed position.

3. The device of claim 2 wherein the tubular attachment comprises an adjustable opening for receiving the catheter or drain.

4. The device of claim 3 wherein the tubular attachment comprises a ratchet mechanism for adjusting the opening to securely fasten the tubular attachment around the catheter or drain.

5. The device of claim 3 wherein the inner surface of the opening comprises teeth for gripping the catheter or drain in a fixed position.

6. The device of claim 3 wherein
the adjustable opening comprises a substantially circular cross section area.

7. The device of claim 1 wherein the connector is made of a plastic type or silicon material.

8. The device of claim 1 wherein the staple is a metal type skin clip comprising means for penetrating skin tissue.

9. The device of claim 1 wherein the staple is made from a biocompatible and/or hypoallergenic material.

10. The device of claim 1 wherein the connector is made from a biocompatible and/or hypoallergenic material.

11. The device of claim 1 wherein part of the connector comprises at least one slot for receiving one leg of said staple to anchor the staple to the patients skin tissue and securing the connector in a fixed position.

12. The device of claim 11 wherein the connector is provided with two slots, a first slot for receiving one leg of said staple and a second slot for receiving a second leg of said staple to anchor the staple to the patients patient's skin tissue.

13. The device of claim 1 wherein the connector is provided with two slots, a first slot for receiving a leg of a first staple and a second slot for receiving a leg of a second staple such that said first and second staples anchor the connector to the patients skin tissue.

14. The device of claim 1 wherein the surgical staple and the connecter are integrally formed.

15. A medical attachment device for securing a catheter or drain to a patient comprising:
a base comprising a layer of adhesive, suitably made of hypoallergenic material, capable of being anchored to the patient's skin tissue through an adhesive mechanism and/or at least one medical staple stapled through a plurality of openings provided in said base; and
said base comprises an embedded connector for attaching to the catheter or drain and securing the catheter or drain in a fixed position.

16. The device of claim 15 wherein the imbedded connecter comprises a channel for receiving the catheter or drain.

17. The device of claim 16 wherein the channel comprises an adjustable opening for receiving the catheter or drain.

18. The device as claimed in claim 16 wherein the inner surface of the channel is coated with an adhesive material for securing the catheter or drain.

19. The device as claimed in claim 16 wherein the channel is substantially tubular shaped.

20. The device as claimed in claim 16 wherein said base comprises two adhesive flaps that adhere to the drain, such that said flaps can be stretched in the opposite direction to each other over said channel, and to fasten to the base to secure the catheter or drain in said channel.

21. The device of claim 20 wherein the inner surface of the flaps are coated with an adhesive material.

22. (canceled)

23. A method for securing a catheter or drain to a patient comprising the steps of:
stapling a staple to a patient's skin tissue to anchor the staple;
coupling the staple to a connector; and
attaching the catheter or drain to the connector to secure the catheter or drain in a fixed position.

24. A medical stapler adapted to use a medical attachment device according to claim 1.

25. A kit comprising a medical stapler according to claim 24 and a medical attachment device for securing a catheter or drain to a patient comprising:
at least one medical staple capable of being anchored to the patient's skin tissue; and
the medical staple is adapted to be coupled to a connector wherein the connector attaches to the catheter or drain to secure the catheter or drain in a fixed position.

26. A medical stapler adapted to use a medical attachment device according to claim 15.

27. A kit comprising a medical stapler according to claim 26 and a medical attachment device for securing a catheter or drain to a patient comprising:
a base comprising a layer of adhesive, suitably made of hypoallergenic material, capable of being anchored to the patient's skin tissue through an adhesive mechanism and/or at least one medical staple stapled through a plurality of openings provided in said base, and
said base comprises an embedded connector for attaching to the catheter or drain and securing the catheter or drain in a fixed position.
